(19) United States
(12) Patent Application Publication
(10) Pub. No.: US 2021/0224742 A1
(43) Pub. Date: Jul. 22, 2021

(54) METHOD FOR CREATING UNDERWRITING DECISION TREE, COMPUTER DEVICE AND STORAGE MEDIUM

(71) Applicant: Ping An Technology (Shenzhen) Co., Ltd., Shenzhen (CN)

(72) Inventors: Zhengbo Shao, Shenzhen (CN); Bin Li, Shenzhen (CN); Jie Chen, Shenzhen (CN); Xue Gao, Shenzhen (CN); Xiangdong Ma, Shenzhen (CN); Jie Ding, Shenzhen (CN); Jie Zhang, Shenzhen (CN)

(21) Appl. No.: 16/096,011

(22) PCT Filed: Sep. 29, 2017
(86) PCT No.: PCT/CN2017/104598

§ 371 Date: Oct. 24, 2018

(30) Foreign Application Priority Data
Jul. 26, 2017 (CN) 201710618080.0

(51) Int. Cl. G06Q 10/10; G06Q 40/08; G06N 5/00; G06N 20/00

(52) U.S. Cl. CPC G06Q 10/10; G06Q 40/08; G16H 10/60; G06N 20/00; G06N 5/003

(57) ABSTRACT

A method for creating an underwriting decision tree includes: acquiring a sample training set including different sample attributes; calculating an entropy value gain that represents an effect of an attribute on an underwriting result of each attribute, according to the underwriting result of a sample of each attribute in the sample training set; taking the attribute with a highest entropy value gain as a current node of the underwriting decision tree, and dividing a sub-attribute corresponding to the attribute with the highest entropy value gain as a next node of the current node; extracting a divided sample training subset of the sub-attribute from the sample training set; determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

This application claims priority to Chinese patent application No. 201710618080.0 entitled “METHOD AND DEVICE FOR CREATING UNDERWRITING DECISION TREE, COMPUTER DEVICE AND STORAGE MEDIUM”, and filed on Jul. 26, 2017, the contents of which is incorporated herein by reference in its entirety.

### TECHNICAL FIELD

The present disclosure relates to the field of insurance technology, and more particularly, to a method and a device for creating an underwriting decision tree, a computer device and a storage medium.

### BACKGROUND

In the field of insurance, it is often necessary to underwrite an insurance policy of a user, to examine whether the corresponding insurance policy can be approved, according to relevant information of an underwriter, such as age, occupation, income, and gender, etc.

At present, the insurance policy of the user is mainly examined manually, based on related information of an insurant, and on work experience of an examiner. However, for an individual whose work experience is not sufficient, it is difficult to examine the insurance policy of the user accurately, without corresponding visual historical data as a reference.

### SUMMARY

According to various embodiments of the invention, a method and a device for creating an underwriting decision tree, a computer device and a storage medium are provided.

A method for creating an underwriting decision tree includes: acquiring a sample training set including different sample attributes; calculating an entropy value gain that represents an effect of an attribute on an underwriting result of each attribute, according to the underwriting result of a sample of each attribute in the sample training set; taking the attribute with a highest entropy value gain as a current node of the underwriting decision tree, and dividing a sub-attribute corresponding to the attribute with the highest entropy value gain as a next node of the current node; extracting a sample training subset of the divided sub-attribute from the sample training set; determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

A device for creating an underwriting decision tree includes: a sample acquisition module, configured to acquire a sample training set including different sample attributes; an entropy value gain calculation module, configured to calculate an entropy value gain that represents an effect of an attribute on an underwriting result of each attribute, according to the underwriting result of a sample of each attribute in the sample training set; a node division module, configured to take the attribute with the highest entropy value gain as a current node, and divide a sub-attribute corresponding to the highest entropy value gain as a next node of the current node; a subset extraction module, configured to extract a sample training subset of the sub-attribute from the sample training set; and a recursion module, configured to determine the sample training subset as the sample training set, and calculate the entropy value gain of the sub-attribute recursively and divide the sub-attribute till the divided sub-attribute of the next node satisfies a preset condition of becoming a leaf node of the underwriting decision tree.

A computer device includes a memory and one or more of processors, and the memory has computer readable instructions stored thereon; following steps are implemented when the computer readable instructions are executed by the one or more of processors: acquiring a sample training set including different sample attributes; calculating an entropy value gain that represents an effect of an attribute on an underwriting result of each attribute, according to the underwriting result of a sample of each attribute in the sample training set; taking the attribute with a highest entropy value gain as a current node of the underwriting decision tree, and dividing a sub-attribute corresponding to the attribute with the highest entropy value gain as a next node of the current node; extracting a sample training subset of the divided sub-attribute from the sample training set; and determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

One or more non-volatile readable storage media have computer readable instructions stored thereon, and following steps are implemented when the computer readable instructions are executed by the one or more of processors: acquiring a sample training set including different sample attributes; calculating an entropy value gain that represents an effect of an attribute on an underwriting result of each attribute, according to the underwriting result of a sample of each attribute in the sample training set; taking an attribute with a highest entropy value gain as a current node of the underwriting decision tree, and dividing a sub-attribute corresponding to the attribute with the highest entropy value gain as a next node of the current node; extracting a sample training subset of the divided sub-attribute from the sample training set; and determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

Details of one or more of embodiments of the invention are presented in following drawings and descriptions. Other characteristics, purposes and advantages of the disclosure will become apparent from the specification, the drawings and the claims.

### BRIEF DESCRIPTION OF DRAWINGS

To make a clearer description of the technical schemes in the embodiments of the invention, drawings used in the embodiments will be briefly introduced, and it is obvious that the drawings in the following description illustrate only some of the embodiments in the invention. For those skilled in the art, other drawings can also be obtained from these drawings without creative work.

FIG. 1 is a flowchart of a method for creating an underwriting decision tree in one embodiment;

FIG. 2 is a flowchart of the method for creating the underwriting decision tree in another embodiment;

FIG. 3 is a flowchart of the method for creating the underwriting decision tree in a further embodiment;

FIG. 4 is a diagram of an application scenario in one embodiment;

FIG. 5 is an exemplary block diagram of a device for creating the underwriting decision tree in one embodiment;

FIG. 6 is an internal structural diagram of a computer device in one embodiment.

### DETAILED DESCRIPTION OF DISCLOSED EMBODIMENTS

To facilitate understanding of the purposes, the technical scheme and the advantages in the present disclosure, the present disclosure will be more fully described below with reference to relevant drawings. It should be understood that the embodiments described herein are used for explaining the present disclosure, rather than for limiting it.

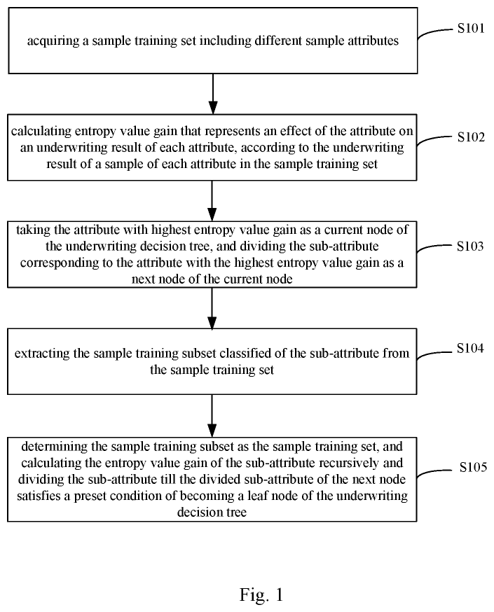

FIG. 1 is a flowchart of a method for creating an underwriting decision tree according to one embodiment of the invention. The method for creating the underwriting decision tree according to one embodiment of the invention is described in detail below with reference to FIG. 1. As shown in FIG. 1, the method includes following steps of S101, S102, S103, S104 and S105.

S101, acquiring a sample training set including different sample attributes.

According to an example of the embodiment, the source of the sample training set includes sample data chosen from historical underwriting records, and it is more instructive for an examiner by taking the sample data chosen from the historical underwriting records as a basis of creating the underwriting decision tree.

In this step, the above attributes include at least two of the follows cases: age, industry risk, anamnesis, and loss ratio, wherein a sub-attribute of age includes young age, elder age, and middle age; a sub-attribute of industry risk includes high, low, and middle; a sub-attribute of anamnesis includes yes and no; and a sub-attribute of loss ratio includes high and low.

A sample training set acquired according to an example of the embodiment is shown in Table (1) below:

TABLE 1

| | Industry | | Loss | Approved | |
|---|---|---|---|---|---|
| Age | risk | Anamnesis | ratio | or not | Count |
| Young | High | No | High | Yes | 640 |
| Elder | High | No | High | No | 1280 |
| Middle | Middle | No | High | No | 600 |
| Young | High | No | Low | Yes | 640 |
| Middle | Low | Yes | High | No | 640 |
| Middle | Low | Yes | Low | Yes | 640 |
| Elder | Low | Yes | Low | No | 640 |
| Young | Middle | No | Low | Yes | 1280 |
| Middle | Middle | Yes | High | No | 1320 |
| Young | Middle | Yes | Low | No | 640 |
| Elder | Middle | No | Low | No | 320 |
| Young | Low | Yes | High | No | 640 |
| Elder | High | Yes | High | No | 320 |
| Middle | Middle | No | Low | Yes | 630 |
| Middle | Middle | No | Low | No | 10 |

Wherein, each age range can be set according to a need of an actual business. According to an example of the embodiment, the age of 0˜25 years old can be set as young age, and the age of 26˜45 years old can be set as middle age, and the age of 46 years and above can be set as elder age.

S102, calculating entropy value gain that represents an effect of the attribute on an underwriting result of each attribute, according to the underwriting result of a sample of each attribute in the sample training set.

According to an example of the embodiment, S102 specifically includes: extracting the underwriting result of the sample of the same attribute from the sample training set, and then calculating the entropy value gain of the attribute according to the underwriting result of the same attribute.

In one embodiment, the underwriting result of S102 includes: whether or not approve an insurance policy, a pass rate of underwriting and a failure rate of underwriting of the corresponding attribute, wherein the entropy value gain can be calculated by following formula:

$${G}_{A} = - ( M \times {log}_{2} M + ( 1 - M ) {log}_{2}^{( 1 - M )} ) - {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ; \qquad ( 1 )$$

Wherein, M represents a total underwriting pass rate in the sample training set, and Aᵢ represents a ratio of the number of sub-attribute i corresponding to an attribute A to the total number of the sample training set, and Bᵢ represents the pass rate of underwriting of the sub-attribute i based on the number of attribute A, and n represents the number of sub-attributes corresponding to the attribute A, and G_{A} represents the calculated entropy value gain of the attribute A.

In an embodiment, an overall decision entropy value of the sample training set can be calculated first according to the sample training set, and then the entropy value of one attribute of the sample training set can be calculated. Then the difference between the decision entropy value and the entropy value of one attribute in the sample training set can be determined as the entropy value gain of the attribute. A significance of the entropy value gain is that it can indicate an influence of the attribute on the underwriting result, the greater the entropy value gain is, the greater the influence is on the underwriting result.

According to an application scenario in one embodiment, the underwriting result of an attribute of age extracted from the above sample training set is shown in Table (2) below:

TABLE 2

| | Underwriting | |
|---|---|---|
| Age | Decision | Count |
| Middle | Pass | 1270 |
| Middle | Fail | 2570 |
| Young | Pass | 2560 |
| Young | Fail | 1280 |
| Elder | Fail | 2560 |
| Elder | Pass | 0 |

According to the above Table (1), it can be derived that:

an overall pass rate

$$M = \frac{3830}{6410 + 3830} ,$$

and an overall failure rate

$$( 1 - M ) = \frac{6410}{6410 + 3830} ;$$

When the above attribute A represents the attribute of age, the sub-attribute i of the attribute A includes the middle age, the young age and the elder age, and it can be derived according to the above Table (1) and Table (2) that:

the ratio of the number of the sub-attribute of young age of the attribute of age to the total number of the sample training set is

$${A}_{i} = \frac{3840}{10240} ,$$

the pass rate of underwriting of the sub-attribute of young age based on the number of the attribute A is

$${B}_{i} = \frac{2560}{1280 + 2560} ,$$

the failure rate of underwriting of the sub-attribute of young age based on the number of the attribute A is

$$( 1 - {B}_{i} ) = \frac{1280}{1280 + 2560} ,$$

and the decision entropy value S_{G} can be calculated as:

$$\begin{matrix}{S}_{G} = - ( {M}_{i} \times {log}_{2} M + ( 1 - M ) \times {log}_{2}^{( 1 - M )} ) \\ = - ( \begin{matrix}\frac{3830}{6410 + 3830} \times {log}_{2} \frac{3830}{6410 + 3830} + \\ \frac{6410}{6410 + 3830} \times {log}_{2} \frac{6410}{6410 + 3830}\end{matrix} ) \\ = 0.9573 ;\end{matrix}$$

and the entropy value of the sub-attribute of young age can be calculated as S_{Ai}:

$$\begin{matrix}{S}_{Ai} = - {B}_{i} \times {log}_{2}^{{B}_{i}} - ( 1 - {B}_{i} ) \times {log}_{2}^{1 - {B}_{i}} ) \\ = - ( \begin{matrix}\frac{2560}{1280 + 2560} \times {log}_{2} \frac{2560}{1280 + 2560} + \\ \frac{1280}{1280 + 2560} \times {log}_{2} \frac{1280}{1280 + 2560}\end{matrix} ) \\ = 0.9138 ;\end{matrix}$$

Similarly, the entropy value of the sub-attribute of middle age can be calculated as 0.9157, and the entropy value of the sub-attribute of elder age can be calculated as 0; and then the entropy value of the attribute A of age can be calculated by Formula (2) below:

$${S}_{A} = {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ; \qquad ( 2 )$$

The entropy value of the attribute A of age can be calculated as:

$$S = \frac{3840}{10240} \times 0.918 \; 3 + \frac{3840}{10240} \times 0.915 \; 7 + \frac{2560}{10240} \times 0 = 0 .6877 ;$$

Again by the above Formula (1), the entropy value gain G_{A} of the attribute of age can be calculated as:

G_{A}=0.9537−0.6877=0.2660.

Similarly, referring to the above Table (1), the entropy value gain of the industry risk, the anamnesis and the loss ratio can be calculated as 0.0176, 0.1726 and 0.0453 respectively.

S103, taking the attribute with highest entropy value gain as a current node of the underwriting decision tree, and dividing the sub-attribute corresponding to the attribute with the highest entropy value gain as a next node of the current node.

The greater the entropy value gain is, the greater the influence is on the underwriting result, since the significance of the entropy value gain is that it can indicate the influence of the attribute on the underwriting result. Determining the attribute with the highest entropy value gain as the current node of the underwriting decision tree helps the examiner to examine and verify the attributes on upper levels of the underwriting decision tree emphatically, and thereby improving the accuracy of underwriting.

According to the application scenarios of the embodiment, for example, when the entropy value gain of the attribute of age calculated by S102 is the highest, the attribute of age is set as the current node of the underwriting decision tree.

S104, extracting a sample training subset of the divided sub-attribute from the sample training set.

According to one example of the embodiment, when the above attribute with the highest entropy value gain is the attribute of age, and the corresponding sub-attributes of the age include the young age, the elder age and the middle age, the sample training subset of the young age sub-attribute extracted according to an application scenario of the embodiment is shown in following Table (3):

TABLE 3

| | Industry | | Loss | Approved | |
|---|---|---|---|---|---|
| Age-Young | risk | Anamnesis | ratio | or not | Count |
| Young | High | No | High | Yes | 640 |
| Young | High | No | Low | Yes | 640 |
| Young | Middle | No | Low | Yes | 1280 |
| Young | Middle | Yes | Low | No | 640 |
| Young | Low | Yes | High | No | 640 |

S105, determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

According to one example of the embodiment, “calculating recursively” in this step refers to determining the corresponding sub-attribute as the attribute A in the above Formula (1), and calculating the entropy value gain of the sub-attribute of the attribute A and dividing branches of the underwriting decision tree according to the extracted sample training subset, till the divided sub-attribute of the next node satisfies the preset condition of becoming the leaf node of the underwriting decision tree.

According to an application scenario of the embodiment with reference to the Table (3), it namely includes processes of determining the above Table (3) as the above sample training set, determining the attribute of young age as the attribute A in the above Formula (1), and calculating the entropy value gain of each attribute of the industry risk, the anamnesis and the loss ratio one by one, calculating them recursively, till the divided sub-attribute of the next node satisfies the preset condition of becoming the leaf node of the underwriting decision tree.

In one embodiment, each sub-attribute of the attribute of age is extracted by S104 and calculated recursively by S105, till the divided sub-attribute of the next node satisfies the preset condition of becoming the leaf node of the underwriting decision tree.

In the embodiment, the attribute with the highest entropy value gain is set as a root node through calculating the entropy value gain of each attribute in the sample training set; and the underwriting decision tree based on each attribute is created, through dividing an attribute of an intermediate node and an attribute of the leaf node of the underwriting decision tree recursively, in order that the examiner can examine and verify the attributes, such as the root node, on the upper levels of the underwriting decision tree emphatically, and provide an underwriting basis for a user, thereby improving the accuracy of underwriting.

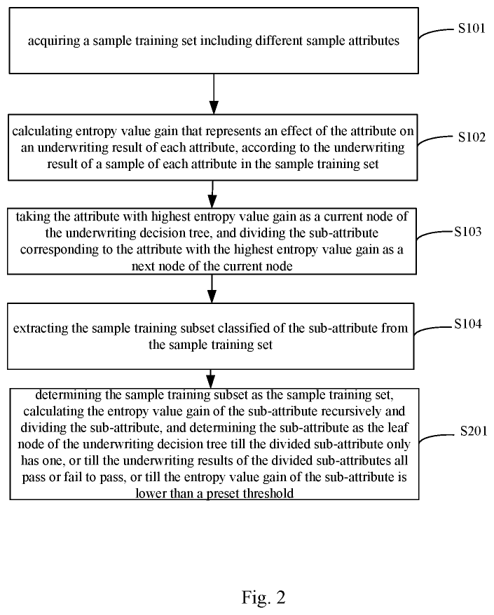

FIG. 2 is a flowchart of the method for creating the underwriting decision tree according to one embodiment of the present invention. As shown in FIG. 2, the method for creating the underwriting decision tree includes the above steps of S101 to S104, and the step S105 further includes the following step S201.

S201, determining the sample training subset as the sample training set, calculating the entropy value gain of the sub-attribute recursively and dividing the sub-attribute, and determining the sub-attribute as the leaf node of the underwriting decision tree till the divided sub-attribute only has one, or till the underwriting results of the divided sub-attributes all pass or fail to pass, or till the entropy value gain of the sub-attribute is lower than a preset threshold.

According to one example of the embodiment, when the entropy value gain of the sub-attribute is lower than the preset threshold, the sub-attribute corresponding to the entropy value gain lower than the preset threshold can be pruned, and an attribute of a previous node of the sub-attribute is determined as the leaf node of the underwriting decision tree.

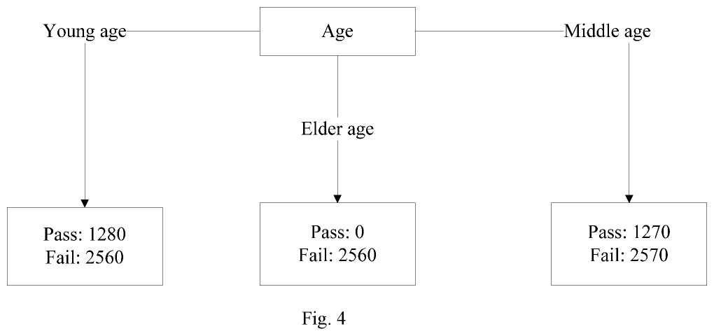

FIG. 4 is an application scenario according to one embodiment of the present invention, and the application scenario of determining the leaf node according to the present embodiment is shown in the FIG. 4, wherein, the sub-attribute of elder age is determined as the leaf node of the underwriting decision tree, when the underwriting results of the leaf node of elder age are all failed. According to another application scenario based on the present embodiment with reference to the above Table (3), for example, when the attributes of the branch of the underwriting decision tree from the root node to the leaf node are in the sequence of age—young age—anamnesis, insurance policies with anamnesis occurred all fail to pass the underwriting, and the insurance policies with no anamnesis occurred all pass the underwriting, therefore, the anamnesis can be determined as the leaf node in the branch of “age—young age—anamnesis” in the underwriting decision tree.

According to another application scenario of the embodiment, for example, when the attributes of the branch of the underwriting decision tree from the root node to the leaf node divided recursively are in the sequence of age—young age—industry risk—anamnesis, wherein no anamnesis is occurred and the sub-attribute of anamnesis only includes the loss ratio, the sub-attribute of loss ratio can be determined as the leaf node of the underwriting decision tree.

According to a further application scenario of the embodiment, when the attributes of the branch of the underwriting decision tree from the root node to the leaf node divided recursively are in the sequence of age—young age—industry risk—anamnesis, wherein no anamnesis is occurred and the entropy value gain of the anamnesis is lower than the preset threshold, the anamnesis can be determined as the leaf node of the underwriting decision tree, alternatively, the leaf node of the anamnesis can be pruned, and its previous node of industry risk can be determined as the leaf node of the underwriting decision tree.

In the embodiment, the sub-attribute with extremely low entropy value gain is pruned, which means to remove the sub-attribute with the extremely low entropy value gain from the underwriting decision tree, thereby the underwriting accuracy of the underwriting decision tree is further improved.

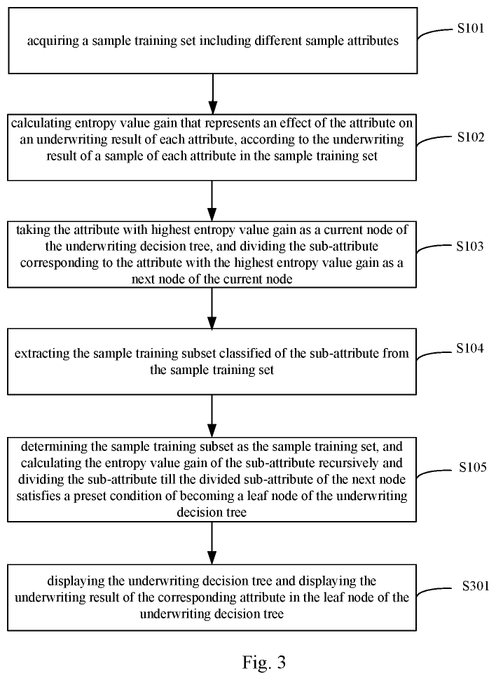

FIG. 3 is a flowchart of the method for creating the underwriting decision tree according to another embodiment of the invention. As shown in FIG. 3, in addition to the above steps S101 to S105, the method for creating the underwriting decision tree further includes the following step S301:

S301, displaying the underwriting decision tree and displaying the underwriting result of the corresponding attribute in the leaf node of the underwriting decision tree.

According to one example of the embodiment, the underwriting result in the step can be the number of the sub-attributes that pass the underwriting and the number of the sub-attributes that are failed, as shown in FIG. 4, alternatively, the underwriting result can be the pass rate and/or the failure rate of the underwriting corresponding to the sub-attribute of the leaf node.

According to one example of the present invention, a method for underwriting automatically by using the underwriting decision tree is provided. This method includes: acquiring each attribute in the insurance policy to be underwritten, matching the attribute acquired with the attribute of each node of the decision tree, and taking the underwriting result corresponding to the leaf node of the underwriting decision tree as the underwriting result of the insurance policy when the attribute of the insurance policy successfully matches with the attribute of the leaf node of the underwriting decision tree.

Wherein, the step of matching the attribute acquired with the attribute of each node of the underwriting decision tree further includes: acquiring the attribute of the current node of the underwriting decision tree; determining the attribute of the insurance policy to be underwritten that is the same as the attribute of the current node as matching successfully; further acquiring the sub-attribute of the attribute of the insurance policy that is successfully matched with the attribute of the current node; querying in the underwriting decision tree for the attribute of the branch that is the same as the sub-attribute acquired; then matching the other attributes of the sub-attribute with the attribute of the intermediate node of the underwriting decision tree, determining the underwriting result of the leaf node as the underwriting result of the insurance policy to be underwritten till the leaf node of the underwriting decision tree is successfully matched.

According to an application scenario of the embodiment, for example, when the branch of the underwriting decision tree from the current node to the leaf node are in the sequence of age—young age—high industry risk, wherein the underwriting results of the attribute of the leaf node of high industry risk are all failed, if the attribute of age in the insurance policy to be underwritten is young age, the anamnesis is occurred, and the industry risk is high, then following steps are implemented sequentially: matching the attribute of age to the sub-attribute of young age in the underwriting decision tree, and acquiring the next node of the young age in the underwriting decision tree as high industry risk, and matching the attribute of high industry risk of the insurance policy to be underwritten to the high industry risk of young age in the underwriting decision tree, and then determining the high industry risk as the leaf node of the underwriting decision tree. And if the underwriting results of the leaf node are all failed, the attribute of anamnesis of the insurance policy to be underwritten does not need to be matched, and the decision that the insurance policy to be underwritten is not approved can be directly made.

According to an example of the embodiment, the numerals of the above steps S101˜S301 are not used to limit the sequence of each step in the present embodiment, and the serial number of each step is only for providing a convenient reference for each step described. As long as the order of executing each step does not affect the logical relation, all possible sequences of steps are regarded as within the protection scope of the disclosure.

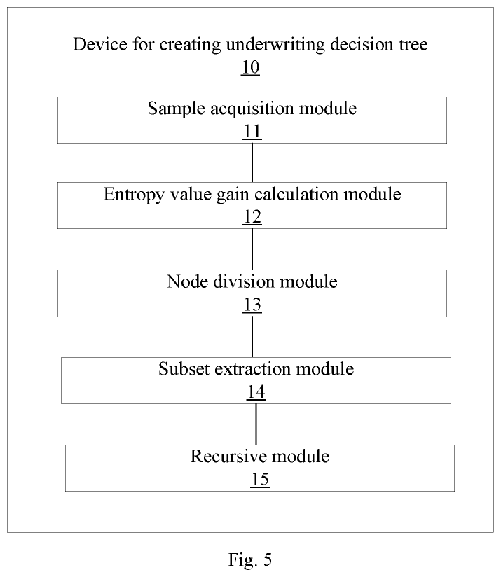

FIG. 5 is an exemplary block diagram of the device for creating the underwriting decision tree according to one embodiment of the present invention. The device for creating the underwriting decision tree according to one embodiment of the present invention will be described with reference to FIG. 5. As shown in FIG. 5, the device for creating the underwriting decision tree 10 includes: a sample acquisition module 11, configured to acquire a sample training set including different sample attributes; an entropy value gain calculation module 12, configured to calculate an entropy value gain that that represents an effect of an attribute on an underwriting result of each attribute, according to the underwriting result of a sample of each attribute in the sample training set; a node division module 13, configured to take an attribute with a highest entropy value gain as a current node, and divide a sub-attribute corresponding to the highest entropy value gain as a next node of the current node; a subset extraction module 14, configured to extract a sample training subset of the sub-attribute from the sample training set; a recursion module 15, configured to determine the sample training subset as the sample training set, and calculate the entropy value gain of the sub-attribute recursively and divide the sub-attribute till the divided sub-attribute of the next node satisfies a preset condition of becoming a leaf node of the underwriting decision tree.

According to an example of the embodiment, the above sample acquisition module is specifically configured to choose the sample data from the historical underwriting records, and it is more instructive for the examiner by taking the sample data chosen from the historical underwriting records as the basis for creating the underwriting decision tree.

In one embodiment, the entropy value gain calculation module 12 is further configured to extract the underwriting results of samples of the same attribute from the sample training set, and then calculate the entropy value gain of the attribute according to the underwriting result of the same attribute.

According to an example of the embodiment, the entropy value gain calculation module 12 is further configured to calculate a total decision entropy value S_{G} of the sample training set first based on the sample training set, then calculate entropy value SA of one attribute in the sample training set, and take the difference between the decision entropy value and the entropy value of the one attribute in the sample training set as the entropy value gain of the attribute. The significance of the entropy value gain is that it can indicate the influence of the attribute on the underwriting result, the greater the entropy value gain is, the greater the influence is on the underwriting result.

Wherein, S_{G}=−(M×log₂ M+(1−M)log₂^{(1-M)}

$${S}_{A} = {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ,$$

wherein, the entropy value of each sub-attribute of attribute A is S_{Ai}:

S_{Ai}=−Bᵢ×log₂^{B}^{ᵢ}−(1−Bᵢ)×log₂^{1-B}^{ᵢ})_{∘}

In one embodiment, the entropy value gain calculation module calculates the entropy value gain by the following formula:

$${G}_{A} = - ( M \times {log}_{2} M + ( 1 - M ) {log}_{2}^{( 1 - M )} ) - {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ;$$

Wherein, M represents a total underwriting pass rate in the sample training set, and Aᵢ represents a ratio of the number of sub-attribute i corresponding to the attribute A to the total number of the sample training set, and Bᵢ represents the pass rate of underwriting of the sub-attribute i based on the number of attribute A, and n represents the number of sub-attributes corresponding to the attribute A, and G_{A} represents the calculated entropy value gain of the attribute A.

Wherein, the attribute includes at least two of the following cases: age, industry risk, anamnesis and loss ratio, wherein the sub-attributes of the attribute of age include young age, elder age and middle age; and the sub-attributes of the attribute of the industry risk include high, low and middle; the sub-attributes of the attribute of the anamnesis include yes and no; the sub-attributes of the attribute of the loss ratio include high and low.

The recursion module 15 is specifically configured to determine the corresponding sub-attribute as the attribute A in the Formula (1), calculate the entropy value gain of the sub-attribute of the attribute A according to the extracted sample training subset, and divide the branch of the underwriting decision tree, till the divided sub-attribute of the next node satisfies the preset condition of becoming the leaf node of the underwriting decision tree.

Since the significance of the entropy value gain is that it can indicate the influence of the attribute on the underwriting result, the greater the entropy value gain is, the greater the influence is on the underwriting result. The above node division module 13 takes the attribute with the highest entropy value gain as the current node of the underwriting decision tree, which helps the examiner to examine and verify the attributes on the upper levels of the underwriting decision tree emphatically, thereby improving the accuracy of underwriting.

According to an example of the embodiment, the recursion module 15 further includes: a first leaf node determining unit, configured to determine the sub-attribute as the leaf node of the underwriting decision tree when the divided sub-attribute has only one; or a second leaf node determining unit, configured to determine the sub-attribute as the leaf node of the underwriting decision tree when the underwriting results of the divided sub-attributes all pass or fail to pass; or a third leaf node determining unit, configured to determine the sub-attribute as the leaf node of the underwriting decision tree, when the entropy value gain of the sub-attribute is lower than the preset threshold.

According to another example of the embodiment, the third leaf node determining unit is further configured to prune the corresponding sub-attribute when the entropy value gain of the sub-attribute is lower than the preset threshold, and to take the last node attribute of the sub-attribute as the leaf node of the underwriting decision tree.

According to an application scenario of the embodiment, for example, when the underwriting results of the leaf node of the sub-attribute of the elder age are all failed, the sub-attribute of the elder age is determined as the leaf node of the underwriting decision tree. According to another application scenario of the embodiment with reference to the above Table (3), for example, when the attributes of the branch of the underwriting decision tree from the root node to the leaf node are in the sequence of age—young age—anamnesis, the insurance policies with anamnesis occurred are all failed, and the insurance policies with no anamnesis occurred all pass the underwriting, therefore, the anamnesis can be determined as the leaf node of the branch of “age—young age—anamnesis” in the underwriting decision tree.

According to another application scenario of the embodiment, for example, when the attributes of the branch of the underwriting decision tree from the root node to the leaf node calculated recursively are in the sequence of age—young age—industry risk—anamnesis, wherein no anamnesis is occurred and the sub-attribute of anamnesis only includes the loss ratio, the sub-attribute of loss ratio can be determined as the leaf node of the underwriting decision tree.

According to a further application scenario of the embodiment, for example, when the attributes of the branch of the underwriting decision tree from the root node to the leaf node divided recursively are in the sequence of age—young age—industry risk—anamnesis, wherein no anamnesis is occurred and the entropy value gain of the anamnesis is lower than the preset threshold, the anamnesis can be determined as the leaf node of the underwriting decision tree, alternatively, the leaf node of anamnesis can be pruned and its previous node of industry risk can be determined as the leaf node of the underwriting decision tree.

According to an example of the embodiment, the device for creating the underwriting decision tree 10 further includes:

A displaying module, configured to display the underwriting decision tree and display the underwriting result of the corresponding attribute in the leaf node of the underwriting decision tree.

According to an example of the embodiment, the display module is specifically configured to display the number of samples that pass and/or fail to pass the underwriting, alternatively, to display the pass rate of underwriting and/or the failure rate of underwriting of the corresponding sub-attributes of the leaf node.

Wherein, the phrase “the first”, “the second” and “the third” in the above first leaf node determining unit, the second leaf node determining unit and the third leaf node determining unit, are used only to distinguish the different leaf node determining units, rather than to define which leaf node determining unit has a higher priority or to have any other limited meaning.

The various modules in the above-described device for creating the underwriting decision tree may be implemented in whole or in part by software, hardware, or the combination thereof. Each of the above modules may be embedded in or be independent to the memory of a terminal in the form of hardware, or be stored in the memory of the terminal in the form of software, so that the processor can call to execute operations corresponding to the above modules. The processor can be a central processing unit (CPU), a microprocessor, a Single Chip Micyoco (SCM), or the like.

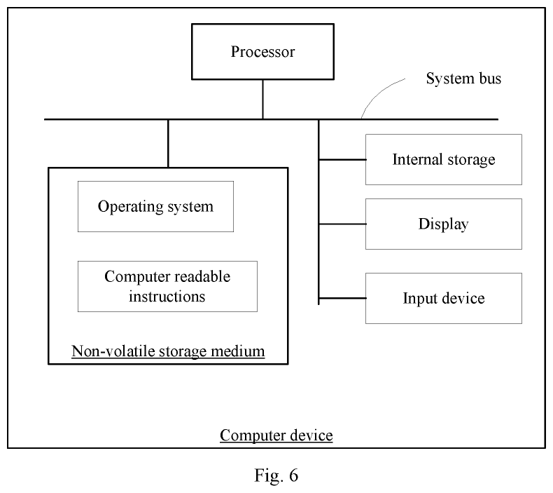

The device for creating the above underwriting decision tree can be implemented in a form of computer readable instructions running on a computer device as shown in FIG. 6.

In one embodiment, a computer device is provided. The internal structure of the computer device may correspond to the structure shown in FIG. 6, namely, the computer device may be a server or a terminal, including a memory and one or more processors. Wherein, the memory stores computer readable instructions, when the computer readable instructions are executed by the processor, following steps are implemented: acquiring a sample training set including different sample attributes; calculating an entropy value gain that represents an effect of an attribute on an underwriting result of each attribute, according to the underwriting result of a sample of each attribute in the sample training set; taking the attribute with a highest entropy value gain as a current node of the underwriting decision tree, and dividing a sub-attribute corresponding to the attribute with the highest entropy value gain as a next node of the current node; extracting a sample training subset of the divided sub-attribute from the sample training set; and determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

In one embodiment, the step of calculating, by the processor, the entropy value gain that represents the effect of the attribute on the underwriting result of each attribute, according to the underwriting result of the sample of each attribute in the sample training set, includes:

Calculating the entropy value gain by the following formula:

$${G}_{A} = - ( M \times {log}_{2} M + ( 1 - M ) {log}_{2}^{( 1 - M )} ) - {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ;$$

Wherein, M represents a total underwriting pass rate in the sample training set, and Aᵢ represents a ratio of the number of sub-attribute i corresponding to the attribute A to the total number of the sample training set, and Bᵢ represents the pass rate of underwriting of the sub-attribute i based on the number of attribute A, and n represents the number of sub-attributes corresponding to the attribute A, and G_{A} represents the calculated entropy value gain of the attribute A.

In an embodiment, the step of determining, by the processor, whether the sub-attribute satisfies the preset condition of becoming the leaf node of the underwriting decision tree includes: determining the sub-attribute as the leaf node of the underwriting decision tree, when the divided sub-attribute only has one; determining the sub-attribute as the leaf node of the underwriting decision tree, when the underwriting results of the divided sub-attribute all pass or fail to pass; determining the sub-attribute as the leaf node of the underwriting decision tree, when the entropy value gain of the sub-attribute is lower than the preset threshold.

In one embodiment, after the step of determining, by the processor, the sample training subset as the sample training set, and calculating the entropy value gain of the sub-attribute recursively and dividing the sub-attribute till the divided sub-attribute of the next node satisfies the preset condition of becoming the leaf node of the underwriting decision tree, the computer readable instructions are further executed by the processor to implement the following steps: displaying the underwriting decision tree and displaying the underwriting result of the corresponding attribute in the leaf node of the underwriting decision tree.

In one embodiment, the attributes include at least two of the following cases: age, industry risk, anamnesis, and loss ratio.

FIG. 6 is a schematic diagram of an internal structure of a computer device according to an embodiment of the present invention, which may be a server. Referring to FIG. 6, the computer device includes a processor, a non-volatile storage medium, an internal memory, an input device, and a display screen connected through a system bus. Wherein the non-volatile storage medium of the computer device can store an operating system and computer readable instructions; when executed, the method for creating the underwriting decision tree in any one of the embodiments of the present invention can be implemented by the processor. For the specific implementing process of the method, reference may be made to the specific content of each embodiment in FIG. 1 to FIG. 4, which will not be repeated here. The processor of the computer device is configured to provide computing and controlling capabilities to support the operation of the entire computer device. The internal memory can store the computer readable instructions, and when the computer readable instructions are executed by the processor, the method for creating the underwriting decision tree can be implemented. The input device of the computer device is used for inputting various parameters, and the display screen of the computer device is used for displaying. It will be understood by those skilled in the art that the structure shown in FIG. 6 is only a block diagram of a part of the structure related to the solution of the present disclosure, and does not constitute a limit to the computer device to which the solution of the present disclosure is applied. The specific computer device may include more or fewer components than those shown in the figures, or may combine some components, or have different component arrangements.

In one embodiment, one or more non-volatile storage media with the computer readable instructions stored thereon are provided, and when the computer readable instructions are executed by the one or more processors, following steps are implemented: acquiring a sample training set including different sample attributes; calculating an entropy value gain that represents an effect of an attribute on an underwriting result of each attribute, according to the underwriting result of a sample of each attribute in the sample training set; taking the attribute with a highest entropy value gain as a current node of the underwriting decision tree, and dividing a sub-attribute corresponding to the attribute with the highest entropy value gain as a next node of the current node; extracting a divided sample training subset of the sub-attribute from the sample training set; determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

In one embodiment, the step of calculating, by the processor, the entropy value gain that represents the effect of an attribute on an underwriting result of each attribute, according to the underwriting result of a sample of each attribute in the sample training set, includes: calculating the entropy value gain by the following formula:

$${G}_{A} = - ( M \times {log}_{2} M + ( 1 - M ) {log}_{2}^{( 1 - M )} ) - {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ;$$

Wherein, M represents a total underwriting pass rate in the sample training set, and Aᵢ represents a ratio of the number of sub-attribute i corresponding to the attribute A to the total number of the sample training set, and Bᵢ represents the pass rate of underwriting of the sub-attribute i based on the number of attribute A, and n represents the number of sub-attributes corresponding to the attribute A, and G_{A} represents the calculated entropy value gain of the attribute A.

In an embodiment, the step of determining, by the processor, whether the sub-attribute satisfies the preset condition of becoming the leaf node of the underwriting decision tree includes: determining the sub-attribute as the leaf node of the underwriting decision tree, when the divided sub-attribute only has one; or determining the sub-attribute as the leaf node of the underwriting decision tree, when the underwriting results of the divided sub-attribute all pass or fail to pass; or determining the sub-attribute as the leaf node of the underwriting decision tree, when the entropy value gain of the sub-attribute is lower than the preset threshold.

In one embodiment, after the step of determining, by the processor, the sample training subset as the sample training set, and calculating the entropy value gain of the sub-attribute recursively and dividing the sub-attribute till the divided sub-attribute of the next node satisfies the preset condition of becoming the leaf node of the underwriting decision tree, the computer readable instructions are further executed by the processor to implement the following steps: displaying the underwriting decision tree and displaying the underwriting result of the corresponding attribute in the leaf node of the underwriting decision tree.

In one embodiment, the attributes include at least two of the following cases: age, industry risk, anamnesis, and loss ratio.

According to an example of the embodiment, all or part of the processes in the above embodiments may be completed by using computer readable instructions to control related hardware, and said programs may be stored in a computer readable storage medium, for example, in the embodiment, the program can be stored in a storage medium of the computer system and executed by at least one processor in the computer system to implement the processes including those in the above embodiments of the method. The storage medium includes, but is not limited to, a magnetic disk, a USB flash drive, an optical disk, a read-only memory (ROM), and the like.

In this embodiment, the underwriting decision tree based on each attribute is created by the following steps: calculating the entropy value gain of each attribute in the sample training set, determining the attribute with the highest entropy value gain as the current node of the underwriting decision tree, and dividing the attribute of the intermediate node and the attribute of the leaf node of the underwriting decision tree recursively, so that the examiner can examine and verify the attributes on the upper levels of the underwriting decision tree emphatically, according to the underwriting decision tree, and can make the underwriting decision directly, according to the underwriting result displayed by the leaf node in the underwriting decision tree, thereby providing the user with a data basis for underwriting, and improving the accuracy and the efficiency of the underwriting.

The technical features of the above-described embodiments can be arbitrarily combined. For simplicity, not all possible combinations of the technical features in the above embodiments are described. However, the combinations shall fall into the scope of the present disclosure as long as there is no contradiction among the combinations of these technical features.

What described above are a plurality of embodiments of the present invention, they are relatively concrete and detailed, but not intended to limit the scope of the present invention. It will be understood by those skilled in the art that various modifications and improvements can be made without departing from the conception of the present invention, and all these modifications and improvements are within the scope of the present invention. The scope of the present invention shall be subject to the claims attached.

1. A method for creating an underwriting decision tree, comprising steps of:
acquiring a sample training set including different sample attributes;
calculating an entropy value gain that represents an effect of an attribute on an underwriting result of each attribute, according to the underwriting result of a sample of each attribute in the sample training set;
taking the attribute with a highest entropy value gain as a current node of the underwriting decision tree, and dividing a sub-attribute corresponding to the attribute with the highest entropy value gain as a next node of the current node;
extracting a sample training subset of the divided sub-attribute from the sample training set; and
determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

2. The method for creating the underwriting decision tree according to claim 1, wherein the step of calculating the entropy value gain that represents the effect of the attribute on the underwriting result of each attribute according to the underwriting result of the sample of each attribute in the sample training set, comprises:
calculating the entropy value gain by a following formula:
$${G}_{A} = - ( M \times {log}_{2} M + ( 1 - M ) {log}_{2}^{( 1 - M )} ) - {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ;$$
wherein, M represents a total underwriting pass rate in the sample training set, and Aᵢ represents a ratio of the number of sub-attribute i corresponding to the attribute A to the total number of the sample training set, and B; represents a pass rate of underwriting of the sub-attribute i based on the number of attribute A, and n represents the number of sub-attributes corresponding to the attribute A, and G_{A} represents the calculated entropy value gain of the attribute A.

3. The method for creating the underwriting decision tree according to claim 1, wherein the step of determining the sample training subset as the sample training set, and calculating the entropy value gain of the sub-attribute recursively and dividing the sub-attribute till the divided sub-attribute of the next node satisfies the preset condition of the leaf node of the underwriting decision tree comprises a sub-step of judging whether the sub-attribute satisfies the preset condition of becoming the leaf node of the underwriting decision tree, and the sub-step comprises:
determining the sub-attribute as the leaf node of the underwriting decision tree, when the divided sub-attribute only has one; or
determining the sub-attribute as the leaf node of the underwriting decision tree, when all the underwriting results of the divided sub-attributes are passed or failed; or
determining the sub-attribute as the leaf node of the underwriting decision tree, when the entropy value gain of the sub-attribute is lower than a preset threshold.

4. The method for creating the underwriting decision tree according to claim 1, wherein after the step of determining the sample training subset as the sample training set, and calculating the entropy value gain of the sub-attribute recursively and dividing the sub-attribute till the divided sub-attribute of the next node satisfies the preset condition of becoming the leaf node of the underwriting decision tree, the method further comprises:
displaying the underwriting decision tree and displaying the underwriting result of the corresponding attribute in the leaf node of the underwriting decision tree.

5. The method for creating the underwriting decision tree according to claim 1, wherein the attributes comprise at least two of the following cases: age, industry risk, anamnesis, and loss ratio.

6.-10. (canceled)

11. A computer device, including a memory and one or more of processors, and the memory having computer readable instructions stored thereon; wherein following steps are implemented when the computer readable instructions are executed by the one or more of processors:
acquiring a sample training set including different sample attributes;
calculating an entropy value gain that represents an effect of an attribute on an underwriting result of each attribute, according to the underwriting result of a sample of each attribute in the sample training set;
taking the attribute with a highest entropy value gain as a current node of the underwriting decision tree, and dividing a sub-attribute corresponding to the attribute with the highest entropy value gain as a next node of the current node;
extracting a sample training subset of the divided sub-attribute from the sample training set; and
determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

12. The computer device according to claim 11, the step of calculating, by the one or more of processors, the entropy value gain that represents the effect of the attribute on the underwriting result of each attribute, according to the underwriting result of the sample of each attribute in the sample training set;
calculating the entropy value gain by the following formula:
$${G}_{A} = - ( M \times {log}_{2} M + ( 1 - M ) {log}_{2}^{( 1 - M )} ) - {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ;$$
wherein, M represents a total underwriting pass rate in the sample training set, and Aᵢ represents a ratio of the number of sub-attribute i corresponding to the attribute A to the total number of the sample training set, and Bᵢ represents a pass rate of underwriting of the sub-attribute i based on the number of attribute A, and n represents the number of sub-attributes corresponding to the attribute A, and G_{A} represents the calculated entropy value gain of the attribute A.

13. The computer device according to claim 11, wherein the step of judging whether the sub-attribute satisfies the preset condition of becoming the leaf node of the underwriting decision tree comprises:
determining the sub-attribute as the leaf node of the underwriting decision tree, when the divided sub-attribute only has one; or
determining the sub-attribute as the leaf node of the underwriting decision tree, when all the underwriting result of the divided sub-attribute are passed or failed; or
determining the sub-attribute as the leaf node of the underwriting decision tree, when the entropy value gain of the sub-attribute is lower than the preset threshold.

14. The computer device according to claim 11, after the step of determining the sample training subset as the sample training set, and calculating the entropy value gain of the sub-attribute recursively and dividing the sub-attribute till the divided sub-attribute of the next node satisfies the preset condition of becoming the leaf node of the underwriting decision tree, the one or more of processors execute the computer readable instructions to implement the following steps:
displaying the underwriting decision tree and displaying the underwriting result of the corresponding attribute in the leaf node of the underwriting decision tree.

15. The computer device according to claim 11, wherein the above attributes comprise at least two of the following cases: age, industry risk, anamnesis, and loss ratio.

16. One or more non-volatile readable storage media, having computer readable instructions stored thereon, wherein following steps are implemented when the computer readable instructions are executed by the one or more of processors:
acquiring a sample training set including different sample attributes;
calculating an entropy value gain that represents an effect of an attribute on an underwriting result of each attribute, according to the underwriting result of a sample of each attribute in the sample training set;
taking an attribute with a highest entropy value gain as a current node of the underwriting decision tree, and dividing a sub-attribute corresponding to the attribute with the highest entropy value gain as a next node of the current node;
extracting a sample training subset of the divided sub-attribute from the sample training set; and
determining the sample training subset as the sample training set, and calculating the entropy value gain of the sub-attribute recursively and dividing the sub-attribute till the divided sub-attribute of the next node satisfies a preset condition of becoming a leaf node of the underwriting decision tree.

17. The one or more non-volatile readable storage media according to claim 16, wherein the step of calculating, by the one or more of processors, the entropy value gain that represents the effect of the attribute on the underwriting result of each attribute, according to the underwriting result of the sample of each attribute in the sample training set, comprises:
calculating the entropy value gain by following formula:
$${G}_{A} = - ( M \times {log}_{2} M + ( 1 - M ) {log}_{2}^{( 1 - M )} ) - {\sum }_{i = 1}^{i = n} {A}_{i} \times ( - {B}_{i} \times {log}_{2}^{{B}_{i}} - ( 1 - {B}_{i} ) \times {log}_{2}^{1 - {B}_{i}} ) ;$$
wherein, M represents a total underwriting pass rate in the sample training set, and Aᵢ represents a ratio of the number of sub-attribute i corresponding to the attribute A to the total number of the sample training set, and Bᵢ represents the pass rate of underwriting of the sub-attribute i based on the number of attribute A, and n represents the number of sub-attributes corresponding to the attribute A, and G_{A} represents the calculated entropy value gain of the attribute A.

18. The one or more non-volatile readable storage media according to claim 16, wherein the step of judging whether the sub-attribute satisfies the preset condition of becoming the leaf node of the underwriting decision tree comprises:
determining the sub-attribute as the leaf node of the underwriting decision tree, when the divided sub-attribute only has one; or
determining the sub-attribute as the leaf node of the underwriting decision tree, when all the underwriting result of the divided sub-attribute are passed or failed; or
determining the sub-attribute as the leaf node of the underwriting decision tree, when the entropy value gain of the sub-attribute is lower than the preset threshold.

19. The one or more non-volatile readable storage media according to claim 16, wherein after the step of determining the sample training subset as the sample training set, and calculating the entropy value gain of the sub-attribute recursively and dividing the sub-attribute till the divided sub-attribute of the next node satisfies the preset condition of becoming the leaf node of the underwriting decision tree, one or more of processors execute the computer readable instructions to implement the following steps:
displaying the underwriting decision tree and displaying the underwriting result of the corresponding attribute in the leaf node of the underwriting decision tree.

20. The one or more non-volatile readable storage media according to claim 16, wherein the above attributes include at least two of the following cases: age, industry risk, anamnesis, and loss ratio.

21. The one or more non-volatile readable storage media according to claim 16, wherein, the above attributes include at least two of the following attributes: age, industry risk, anamnesis, and reimbursement rate.

22. The one or more non-volatile readable storage media according to claim 17, wherein, the above attributes include at least two of the following attributes: age, industry risk, anamnesis, and reimbursement rate.

23. The method for creating the underwriting decision tree according to claim 2, wherein, the attributes comprise at least two of following attributes: age, industry risk, anamnesis, and reimbursement rate.

24. The method for creating the underwriting decision tree according to claim 3, wherein, the attributes comprise at least two of following attributes: age, industry risk, anamnesis, and reimbursement rate.

25. The method for creating the underwriting decision tree according to claim 4, wherein, the attributes comprise at least two of following attributes: age, industry risk, anamnesis, and reimbursement rate.
